(19) United States
(12) Patent Application Publication
(10) Pub. No.: US 2012/0252058 A1
(43) Pub. Date: Oct. 4, 2012

(54) System and Method for the Assessment of Biological Particles in Exhaled Air

(75) Inventors: Jeffrey Cohen, Pittsburgh, PA (US); Shona Stewart, Pittsburgh, PA (US); Oksana Klueva, Pittsburgh, PA (US)

(73) Assignee: ChemImage Corporation, Pittsburgh, PA (US)

(21) Appl. No.: 13/432,938

(22) Filed: Mar. 28, 2012

Related U.S. Application Data
(60) Provisional application No. 61/468,627, filed on Mar. 29, 2011.

(51) Int. Cl. G01N 21/64; C12M 1/34

(52) U.S. Cl. 435/34; 435/288.7

(57) ABSTRACT

A system and method for the deposition and analysis of a sample comprising exhaled particles. The sample may be deposited on a substrate and regions of interest comprising exhaled particles may be targeted using autofluorescence. Regions of interest comprising exhaled particles may then be interrogated to thereby generate a spectroscopic data set. This spectroscopic data set may be compared to a reference data set associated with at least one of the following: a known disease state, a known metabolic state, a known inflammatory state, a known immunologic state, a known infectious state, and combinations thereof.

### RELATED APPLICATIONS

This Application claims priority to U.S. Patent Application No. 61/468,627, entitled “System and Method for Particle Monitoring in Medical Applications,” filed on Mar. 29, 2011, which is hereby incorporated by reference in its entirety.

### BACKGROUND

The biochemical composition of a biological sample may comprise a complex mix of biological molecules including, but not limited to, proteins, nucleic acids, lipids, and carbohydrates. A biological sample may comprise a cell, tissue, and/or bodily fluid. Cells are a basic unit of life. The body of an individual human is made up of many trillions of cells, the overwhelming majority of which, have differentiated to form tissues and cell populations of various discrete types. Cells in a healthy human often exhibit physical and biochemical features that are characteristic of the discrete cell or tissue type. Such features can include the size and shape of the cell, its motility, its mitotic status, its ability to interact with certain chemical or immunological reagents, and other observable characteristics.

The field of cytology involves microscopic analysis of cells to evaluate their structure, function, formation, origin, biochemical activities, pathology, and other characteristics. Known cytological techniques include fluorescent and visible light microscopic methods, alone or in conjunction with use of various staining reagents (e.g., hemotoxylin and eosin stains), labeling reagents (e.g., fluorophore-tagged antibodies), or combinations thereof.

Cytological analyses are most commonly performed on cells obtained from samples removed from the body of a mammal. In vivo cytological methods are often impractical owing, for example, to relative inaccessibility of the cells of interest and unsuitability of staining or labeling reagents for in vivo use. Cells are commonly obtained for cytological analysis by a variety of methods. By way of examples, cells can be obtained from a fluid that contacts a tissue of interest, such as a natural bodily fluid (e.g., blood, urine, lymph, sputum, peritoneal fluid, pleural fluid, or semen) or a fluid that is introduced into a body cavity and subsequently withdrawn (e.g., bronchial lavage, oral rinse, or peritoneal wash fluids). Cells can also be obtained by scraping or biopsying a tissue of interest. Cells obtained in one of these ways can be washed, mounted, stained, or otherwise treated to yield useful information prior to microscopic analysis. Information obtained from cytological analysis can be used to characterize the status of one or more cells in a sample.

Various types of spectroscopy and imaging may be explored for analysis of biological samples. Raman spectroscopy is based on irradiation of a sample and detection of scattered radiation, and it can be employed non-invasively to analyze biological samples in situ. Thus, little or no sample preparation is required. Raman spectroscopy techniques can be readily performed in aqueous environments because water exhibits very little, but predictable, Raman scattering. It is particularly amenable to in vivo measurements as the powers and excitation wavelengths used are non-destructive to the tissue and have a relatively large penetration depth.

Chemical imaging is a reagentless tissue imaging approach based on the interaction of light with tissue samples. The approach yields an image of a sample wherein each pixel of the image is the spectrum of the sample at the corresponding location. The spectrum carries information about the local chemical environment of the sample at each location. For example, Raman chemical imaging (RCI) has, a spatial resolving power of approximately 250 nm and can potentially provide qualitative and quantitative image information based on molecular composition and morphology.

Instruments for performing spectroscopic (i.e. chemical) analysis typically comprise an illumination source, image gathering optics, focal plane array imaging detectors and imaging spectrometers. In general, the sample size determines the choice of image gathering optic. For example, a microscope is typically employed for the analysis of sub-micron to millimeter spatial dimension samples. For larger objects, in the range of millimeter to meter dimensions, macro lens optics are appropriate. For samples located within relatively inaccessible environments, flexible fiberscope or rigid borescopes can be employed. For very large scale objects, such as planetary objects, telescopes are appropriate image gathering optics.

For detection of images formed by the various optical systems, two-dimensional, imaging focal plane array (FPA) detectors are typically employed. The choice of FPA detector is governed by the spectroscopic technique employed to characterize the sample of interest. For example, silicon (Si) charge-coupled device (CCD) detectors or CMOS detectors are typically employed with visible wavelength fluorescence and Raman spectroscopic imaging systems, while indium gallium arsenide (InGaAs) FPA detectors are typically employed with near-infrared spectroscopic imaging systems.

Spectroscopic imaging of a sample can be implemented by one of two methods. First, a point-source illumination can be provided on the sample to measure the spectra at each point of the illuminated area. Second, spectra can be collected over the an entire area encompassing the sample simultaneously using an electronically tunable optical imaging filter such as an acousto-optic tunable filter (AOTF), a multi-conjugate tunable filter (MCF), or a liquid crystal tunable filter (LCTF). Here, the organic material in such optical filters is actively aligned by applied voltages to produce the desired bandpass and transmission function. The spectra obtained for each pixel of such an image thereby forms a complex data set referred to as a hyperspectral image which contains the intensity values at numerous wavelengths or the wavelength dependence of each pixel element in this image.

Assessing biological samples may require obtaining the spectrum of a sample at different wavelengths. Conventional spectroscopic devices operate over a limited range of wavelengths due to the operation ranges of the detectors or tunable filters possible. This enables analysis in the Ultraviolet (UV), visible (VIS), infrared (IR), near infrared (NIR), short wave infrared (SWIR) mid infrared (MIR) wavelengths and to some overlapping ranges. These correspond to wavelengths of about 180-380 nm (UV), 380-700 nm (VIS), 1000-2500 nm (IR), 700-2500 nm (NIR), 850-1700 nm (SWIR) and 2500-25000 nm (MIR).

Spectroscopic techniques may be of particular use in the analysis of dried droplets of bodily fluids because of the influence of constituents of the droplet on the spatial pattern of drying. Due to the properties associated with drying, imaging can determine more specific information about specific molecular families.

Spectroscopic techniques may hold potential for detecting moieties at very small concentrations. In this case, a change in the molecular environment, which essentially amplifies the signal, is detected, as opposed to a low concentration molecule. Analysis may be focused on signals correlated with a history of exposure on different time scales. These signals may manifest themselves through changes in molecular concentration, or structural changes that occur in molecules in a fluid sample such as sputum which may be expelled by exhalation (breathing or coughing) of a patient.

Spectroscopic techniques may also hold potential for assessing a variety of particles that may be exhaled by a patient. If a patient has cough-like symptoms, particles dislodged could contain potentially infectious material such as microbes or viruses, or even cells and cellular debris. These cells and cellular debris may be indicative of disease states including malignancy. Particles recovered via exhalation may indicate relevant things about the physiology of a patient, including response to treatment for pneumonia, and the level of inflammation of the lungs. There exists a need for a system and method for the analysis of samples of exhaled particles for determining a patient's physiological characteristics such as a disease state, a metabolic state, an inflammatory state, an immunologic state, an infectious state, and combinations thereof.

The current state of the art for particle assessment is to use an optical counter followed by performing a polymerase chain reaction (PCR). This process is time consuming and expensive, and the complexity of its execution is dependent on what type of particle is under analysis. The system and method of the present disclosure overcome the limitations of the prior art and provide for an accurate and reliable approach to depositing and assessing a sample of exhaled particles.

### SUMMARY

In one embodiment, the present disclosure provides for a system and method for depositing and analyzing a sample comprising exhaled particles. A sample may be deposited onto a substrate. At least one autofluorescence image representative of the sample may be generated and analyzed to target regions of interest of the sample which exhibit autofluorescence characteristic of exhaled particles. Regions of interest may be interrogated to generate a test data set. The test data set may comprise at least one of: a hyperspectral image, a spectrum, and combinations thereof. The test data set may be analyzed to classify the sample as being associated with at least one physiological condition.

In another embodiment, the present disclosure provides for a system comprising a processor and a non-transitory processor-readable storage medium in operable communication with the processor, wherein the storage medium contains one or more programming instructions. In one embodiment, one or more programming instructions, when executed, may cause the processor to generate at least one autofluorescence image representative of the sample, analyze the autofluorescence image to target regions of interest, and interrogate regions of interest to generate a test data set. The test data set may comprise at least one of: a hyperspectral image, a spectrum, and combinations thereof. The test data set may be analyzed to classify the sample as being associated with at least one physiological condition.

The present disclosure overcomes the limitations of the prior art by incorporating a pre-screening process for determining the optimal regions of a sample which can then be interrogated using hyperspectral imaging. Such an invention combines the benefit of rapid data analysis associated with autofluorescence with the material specific benefits of hyperspectral imaging. The system and method disclosed herein therefore hold potential for rapid, accurate, and reliable interrogation of samples that may be used to assess physiological characteristic of a sample.

### BRIEF DESCRIPTION OF THE DRAWINGS

The accompanying drawings, which are included to provide further understanding of the disclosure and are incorporated in and constitute a part of this specification, illustrate embodiments of the disclosure and, together with the description, serve to explain the principles of the disclosure.

In the drawings:

FIG. 1 depicts an illustrative embodiment of a method of the present disclosure.

FIG. 2A depicts an illustrative embodiment of a fiber array spectral translator device.

FIG. 2B depicts an alternate illustrative embodiment of a fiber array spectral translator device.

FIG. 3A represents division of a sampling space into a grid.

FIG. 3B illustrates the application of a threshold to screening an image based on optimal sensitivity.

FIG. 3C illustrates the identification of optimal regions of interest (ROls) for wide-field RCI of particles.

FIG. 3D illustrates wide-field RCI over an optimal ROI.

FIG. 4A illustrates a brightfield reflectance image of a bladder cell.

FIG. 4B illustrates a dispersive spectrum of a bladder cell.

FIG. 5 illustrates spectra associated with various microorganisms.

FIG. 6A illustrates a brightfield reflectance image of a. Single C. parvum oocyst.

FIG. 6B illustrates a Raman chemical image of a Single C. parvum oocyst.

FIG. 6C illustrates an Image spectrum of a Single C. parvum oocyst.

FIG. 7A represents a brighfield reflectance image of a sample comprising Methicillin Resistant Staphylococcus Aureus.

FIG. 7B illustrates an average spectrum representative of Methicillin Resistant Staphylococcus Aureus.

FIG. 8A represents a brighfield reflectance image of a sample comprising Methicillin Sensitive Staphylococcus Aureus.

FIG. 8B illustrates an average spectrum representative of Methicillin Sensitive Staphylococcus Aureus.

FIG. 9 represents principle component analysis of Methicillin Sensitive Staphylococcus Aureus and Methicillin Resistant Staphylococcus Aureus.

### DETAILED DESCRIPTION

Reference will now be made in detail to the embodiments of the present disclosure, examples of which are illustrated in the accompanying drawings. Wherever possible, the same reference numbers will be used throughout the drawings to refer to the same or like parts.

The present disclosure provides for a system and method for the deposition and analysis of samples comprising exhaled particles. The various embodiments discussed herein hold potential for particle monitoring in a medical setting and may be configured for automatic deposition and/or analysis.

In one embodiment, a system and method of the present disclosure may be configured for operation in conjunction with a medical apparatus such as a medical ventilator. Medical ventilators operate to push air into the lungs of a patient and to remove air from the lungs of a patient. A ventilator may also control the mixture of gases and pressures which the lungs experience. In another embodiment, a system and method of the present disclosure may be configured for operation in conjunction with at least one of: a spirometer, an oxygen mask, a nasal cannula, a mouth piece, and combinations thereof.

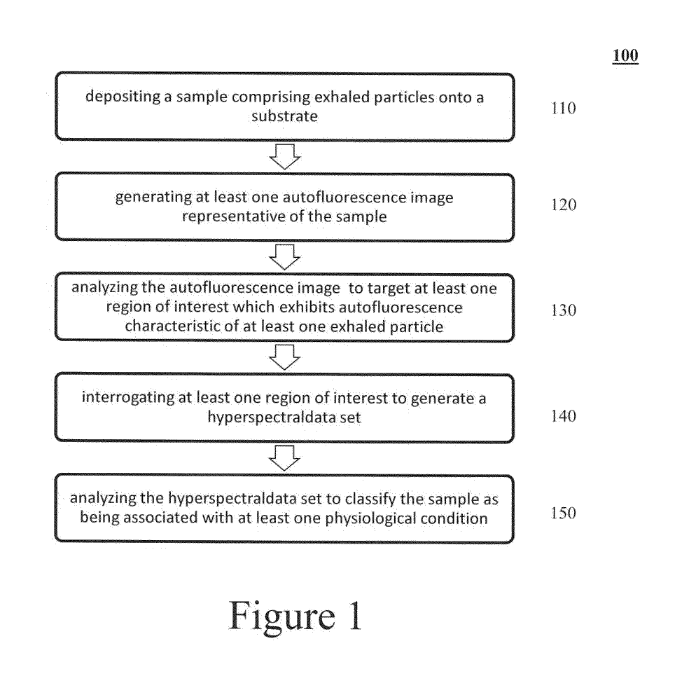

In one embodiment, the present disclosure provides for a method 100, illustratively depicted by FIG. 1. The method 100 may comprise depositing a sample comprising exhaled particles onto a substrate in step 110. In one embodiment, this substrate may comprise a filter medium. In another embodiment, a substrate may comprise a tape, a slide, or another suitable deposition surface.

In one embodiment, deposition of a sample onto a substrate may be accomplished using ultrasonic deposition, electrostatic, electro spray, and inertial impaction of the sample onto the substrate. In one embodiment, the substrate may be housed in an aerosol collection device. In such a configuration the method 100 may further comprise introducing the sample into the aerosol collection device. The present disclosure contemplates that this introduction may occur in various ways. In one embodiment, a patient may breathe into a device operatively coupled to the aerosol collection device. This may be achieved using an oxygen mask, a mouth piece, or another device. As discussed herein, the deposition of exhaled particles may also occur while a patient is using a medical apparatus. In such an embodiment, all or a portion of the exhaled breath may be directed to the aerosol collection device for deposition of a sample. The method 100 may also comprise the transfer of a sample from an aerosol collection device to a hyperspectral imaging device. In one embodiment, this transfer may be automated. Automation may be accomplished by utilizing a tube, mouthpiece or other device. In another embodiment, the transfer may be manual by a user.

In one embodiment, the present disclosure contemplates that the sample may be deposited while a patient is operatively coupled to a medical device. This medical device may include, but is not limited to, a medical ventilator, a spirometer, an oxygen mask, a nasal cannula, a mouth piece, and combinations thereof.

In step, 120 at least one autofluorescence image of the sample may be generated. In one embodiment, generating an autofluorescence image may further comprise illuminating a sample to thereby generate a first plurality of interacted photons. The first plurality of interacted photons may be filtered and detected to generate an autofluorescence image.

The autofluorescence image may be analyzed in step 130 to target regions of interest which exhibit autofluorescence characteristic of exhaled particles. In one embodiment, this analysis may further comprise visual inspection by a user. In step 140 regions of interest may be interrogated to generate a test data set. The test data set may comprise at least one of: a hyperspectral image, a spectrum, and combinations thereof. In one embodiment, generating a test data set may further comprise illuminating a region of interest to thereby generate a second plurality of interacted photons, filtering the second plurality of interacted photons, and detecting the second plurality of interacted photons to generate a test data set. The test data set may comprise at least one of: a hyperspectral image, a spectrum, and combinations thereof. In one embodiment, a test data set may comprise at least one of: a fluorescence test data set, a Raman test data set, an infrared test data set, a visible test data set, an ultraviolet test data set, a LIBS test data set, and combinations thereof. In one embodiment, a plurality of regions of interest may be interrogated. These regions of interest may be interrogated simultaneously or sequentially.

In one embodiment, the illumination of the sample may be accomplished using a substantially monochromatic light source. In another embodiment, the illumination of the sample may be accomplished using a mercury arc lamp.

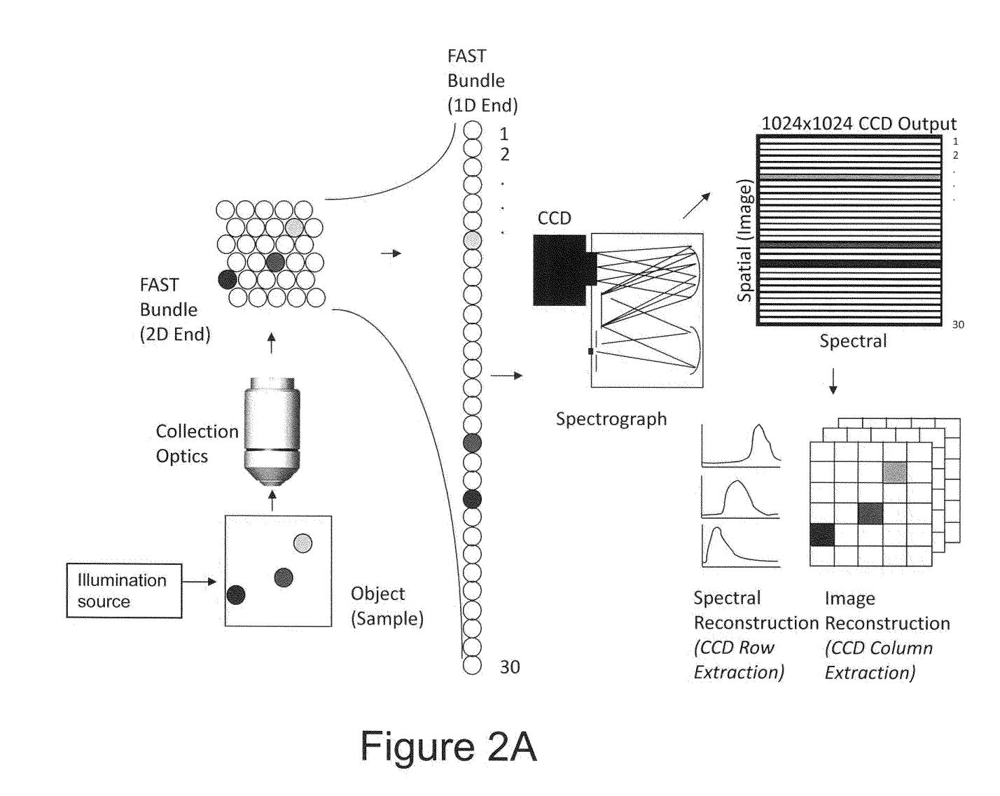

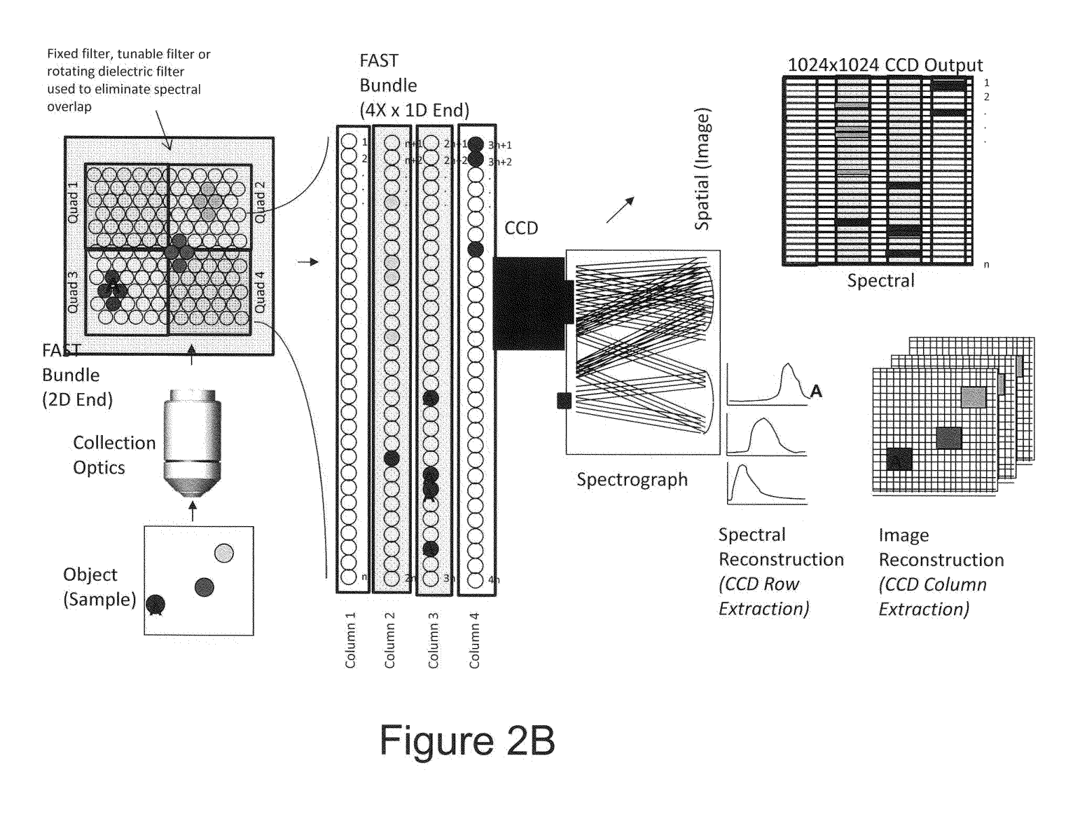

In one embodiment, the method 100 may further comprise passing at least one plurality of interacted photons through a fiber array spectral translator (FAST) device. A FAST device may comprise a two-dimensional array of optical fibers drawn into a one-dimensional fiber stack so as to effectively convert a two-dimensional field of view into a curvilinear field of view, and wherein the two-dimensional array of optical fibers is configured to receive the photons and transfer the photons out of the fiber array spectral translator device and to at least one of: a spectrometer, a filter, a detector, and combinations thereof. Embodiments of FAST devices contemplated by the present disclosure are illustrated by FIG. 2A and FIG. 2B. FIG. 2A is illustrative of a traditional FAST configuration. FIG. 2B is illustrative of a spatially and spectrally parallelized FAST configuration. As disclosed in FIG. 2B, a FAST device may be configured to comprise a two-dimensional array of optical fibers drawn into a one-dimensional fiber stack so as to effectively convert a two-dimensional field of view into a curvilinear field of view, and wherein the two-dimensional array of optical fibers is configured to receive the photons and transfer the photons out of the fiber array spectral translator device and to a spectrograph through the one-dimensional fiber stack wherein the one-dimensional fiber stack comprises at least two columns of fibers spatially offset in parallel at the entrance slit of the spectrograph.

The FAST device can provide faster real-time analysis for rapid detection, classification, identification, and visualization of, for example; particles in pharmaceutical formulations. FAST technology can acquire a few to thousands of full spectral range, spatially resolved spectra simultaneously. This may be done by focusing a spectroscopic image onto a two-dimensional array of optical fibers that are drawn into a one-dimensional distal array with, for example, serpentine ordering. The one-dimensional fiber stack may be coupled to an imaging spectrometer, a detector, a filter, and combinations thereof. Software may be used to extract the spectral/spatial information that is embedded in a single CCD image frame.

One of the fundamental advantages of this method over other spectroscopic methods is speed of analysis. A complete spectroscopic imaging data set can be acquired in the amount of time it takes to generate a single spectrum from a given material. FAST can be implemented with multiple detectors. Color-coded FAST spectroscopic images can be superimposed on other high-spatial resolution gray-scale images to provide significant insight into the morphology and chemistry of the sample.

The FAST system allows for massively parallel acquisition of full-spectral images. A FAST fiber bundle may feed optical information from its two-dimensional non-linear imaging end (which can be in any non-linear configuration, e.g., circular, square, rectangular, etc.) to its one-dimensional linear distal end. The distal end feeds the optical information into associated detector rows. The detector may be a CCD detector having a fixed number of rows with each row having a predetermined number of pixels. For example, in a 1024-width square detector, there will be 1024 pixels (related to, for example, 1024 spectral wavelengths) in each of the 1024 rows.

The construction of the FAST array requires knowledge of the position of each fiber at both the imaging end and the distal end of the array. Each fiber collects light from a fixed position in the two-dimensional array (imaging end) and transmits this light onto a fixed position on the detector (through that fiber's distal end).

Each fiber may span more than one detector row, allowing higher resolution than one pixel per fiber in the reconstructed image. In fact, this super-resolution, combined with interpolation between fiber pixels (i.e., pixels in the detector associated with the respective fiber), achieves much higher spatial resolution than is otherwise possible. Thus, spatial calibration may involve not only the knowledge of fiber geometry (i.e., fiber correspondence) at the imaging end and the distal end, but also the knowledge of which detector rows are associated with a given fiber.

A test data set may be analyzed in step 150 to classify the sample as being associated with at least one physiological condition. In one embodiment, analyzing the test data set may further comprise comparing the test data set to at least one reference data set associated with at least one of: a known disease state, a known metabolic state, a known inflammatory state, a known immunologic state, a known infectious state, and combinations thereof. This comparison may be achieved by applying one or more chemometric techniques. The chemometric technique may comprise at least one of: principle component analysis, linear discriminant analysis, partial least squares discriminant analysis, maximum noise fraction, blind source separation, band target entropy minimization, cosine correlation analysis, classical least squares, cluster size insensitive fuzzy-c mean, directed agglomeration clustering, direct classical least squares, fuzzy-c mean, fast non negative least squares, independent component analysis, iterative target transformation factor analysis, k-means, key-set factor analysis, multivariate curve resolution alternating least squares, multilayer feed forward artificial neural network, multilayer perception-artificial neural network, positive matrix factorization, self modeling curve resolution, support vector machine, window evolving factor analysis, and orthogonal projection analysis.

In one embodiment, the present disclosure provides for a method of assessing malfunction of such a closed-loop breathing system. In the event of a system failure, a particle could be released to the gas introduced into a patient. This particle may then be detected using a method disclosed herein and act as a trigger to indicate system failure. Identification of the particle may also be used to aid in the repair or improvement of the system.

In one embodiment, the present disclosure also provides for a storage medium containing machine readable program code, which, when executed by a processor, causes the processor to perform a method of the present disclosure. In one embodiment, the processor may perform the method 100 of FIG. 1. In such an embodiment, the processor may be configured to perform the following: deposit a sample comprising exhaled particles onto a substrate; generate at least one autofluorescence image representative of the sample; analyze the autofluorescence image to target at least one region of interest which exhibits autofluorescence characteristic of at least one exhaled particle; interrogate at least one region of interest to generate a test data set; and analyze the test data set to classify the sample as being associated with at least one physiological condition.

In one embodiment, the storage medium, when executed by a processor to analyze a test data set, may further cause the processor to compare the test data set to at least one reference data set associated with at least one of: a known disease state, a known metabolic state, a known inflammatory state, a known immunologic state, a known infectious state, and combinations thereof.

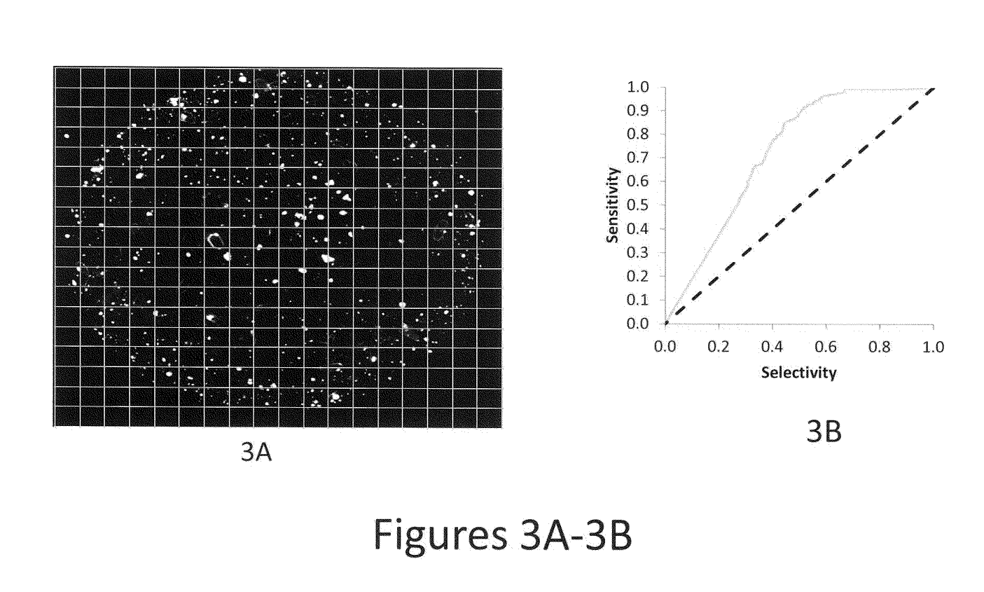

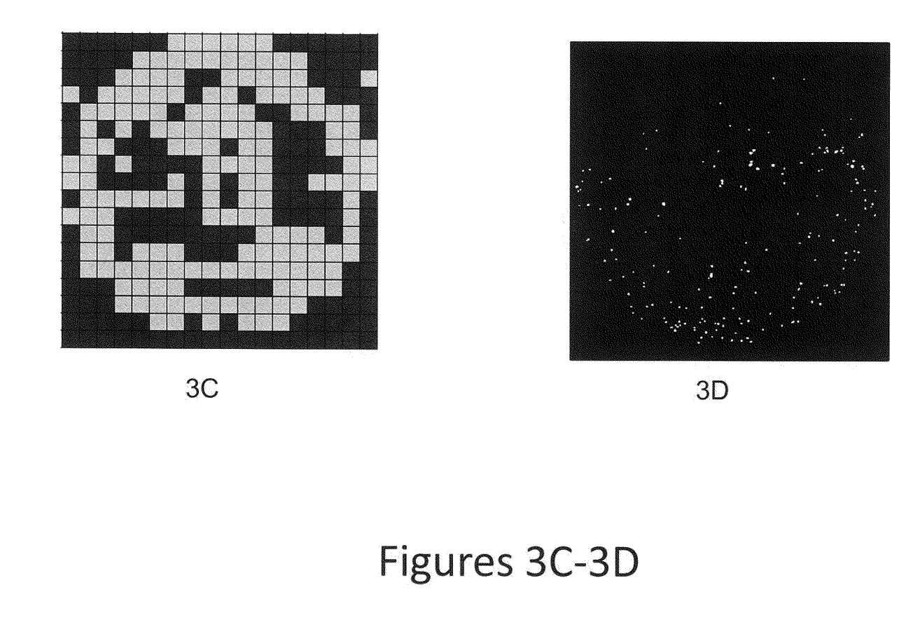

Embodiments of the present disclosure hold potential for assessment of various types of particles that may be present in a sample exhaled by a patient, and deposited onto a substrate. FIGS. 3A-3D illustrates the detection capabilities of particles in an aerosol sample after deposition onto a substrate. FIGS. 3A-3D are illustrative of autofluoroescence targeting and Raman interrogation of regions of interest of a of Rhinocort Aqua® nasal spray sample. Similar to exhaled particle samples, nasal spray suspensions are typically contain sparse particle populations and are dried onto a substrate or filtered through a membrane filter before analysis. Nasal spray suspensions intended for a spectroscopy confirmation step typically include a drying process post sample actuation.

As illustrated by FIG. 3A, in order to efficiently utilize the test data collected, a sampling space may be divided into a grid. FIG. 3B illustrates the application of a threshold to screening an image based on optimal sensitivity. FIG. 3C illustrates the identification of optimal regions of interest (ROIs) for wide-field RCI of particles. If the rapid screening modality registers a detection event inside of the grid, an RCI measurement will be performed for confirmation of particles. FIG. 3D illustrates wide-field RCI over an optimal ROI.

A sample may be divided into a plurality of regions (using a grid or similar format) for mapping locations in the sample. An autofluorescence image of a sample may be generated. Because certain particles of interest will autofluoresce and other particles will not, this autofluorescence image holds potential for indicating areas of a sample where there is a high probability of locating particles of interest. These areas of interest may be targeted and interrogated to generate a test data set. In one embodiment, the test data set may comprise a Raman hyperspectral image that can be used to ascertain information about the particles present in the sample. Such an approach yields spatially accurate spectroscopic information and is well suited for assessment of complex mixtures.

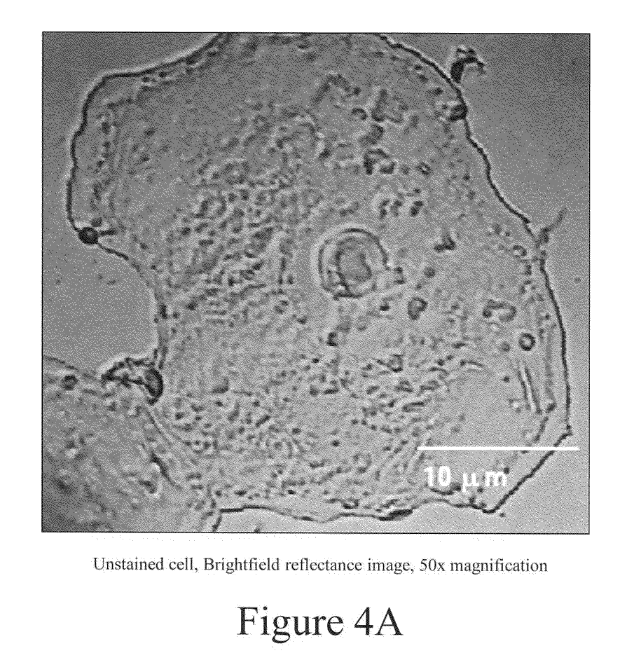

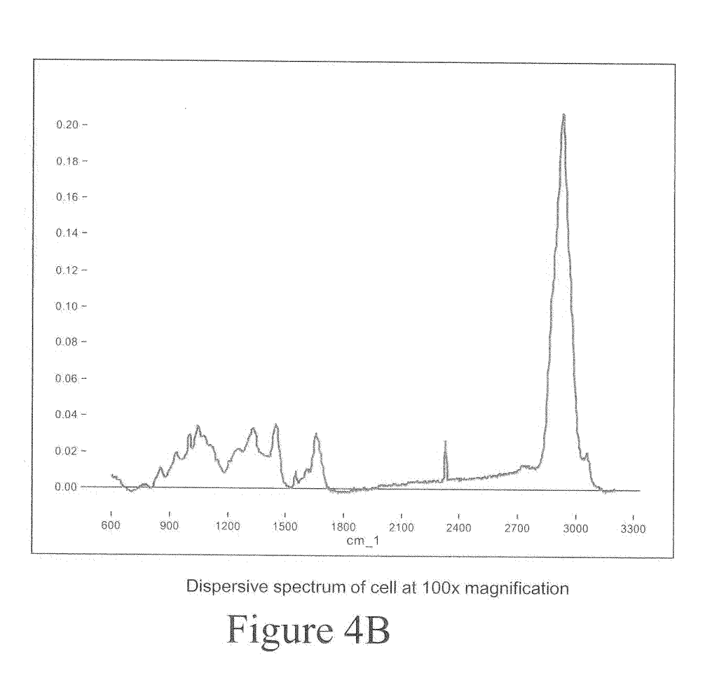

FIGS. 4A-4B illustrate a brightfiled image and a dispersive spectrum of a single bladder cell obtained from a urine sample. Although the cell illustrated is a bladder cell, the system and method may be similarly applied to cells found in exhaled particles such as lung cells and sputum. The present disclosure contemplates that a comparison of test data sets and reference datasets associated with cells hold potential for revealing physiological characteristics such as a disease state, a metabolic state, an inflammatory state, an immunologic state, an infectious state, and combinations thereof.

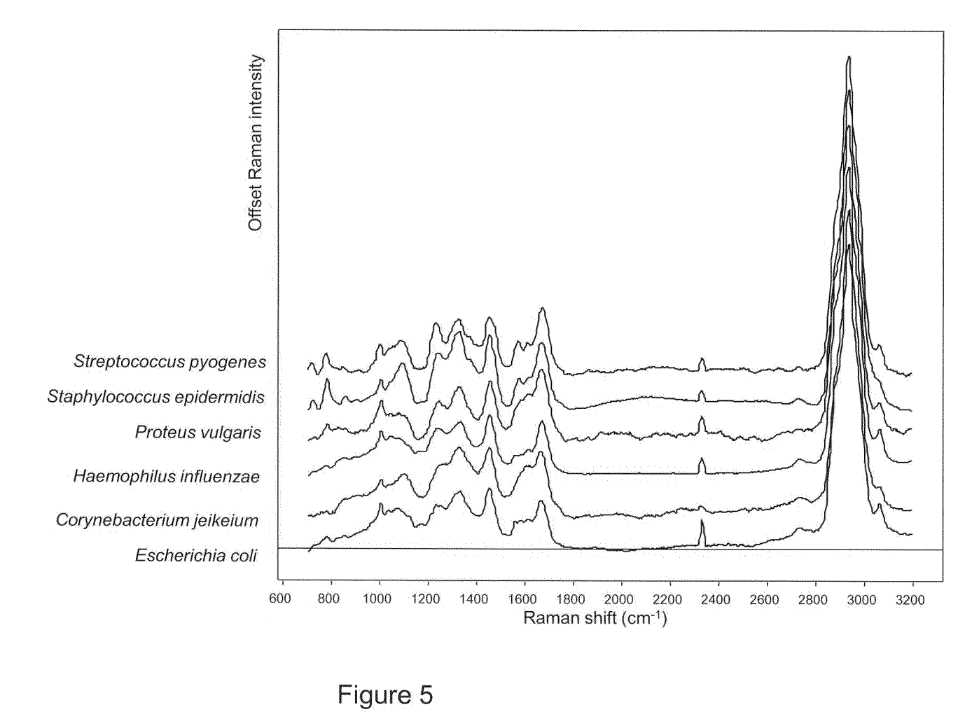

In addition to analyzing cells and cellular debris, a Method of the present disclosure may also be used to detect a variety of microorganisms. FIG. 5 is illustrative of the differences in dispersive spectra of various microorganisms that can be found in the human mouth, nasal passage and GI tract. Examples of pathogens that can be assessed using the methods described herein include bacteria (including eubacteria and archaebacteria), eukaryotic microorganisms (e.g., protozoa, fungi, yeasts, and molds) viruses, and biological toxins (e.g., bacterial or fungal toxins or plant lectins). Specific examples of such pathogens include protozoa of the genus Cryptosporidium, protozoa of the genus Giardia, bacteria of genera such as Escherichia, Escherichia coli, Escherichia coli 157, Yersinia, Francisella, Brucella, Clostridium, Burkholderia, Chlamydia, Coxiella, Rickettsia, Vibrio, Enterococcus, Staphylococcus, Staphylococcus, methicillin-resistant staphylococcus (MRSA), Enterobacter, Cognebacterium, Pseudomonas, Acinetobacter, Klebsiella, and Serratia. Assessable organisms include at least Escherichia coli, Yersinia pestis. Francisella tularensis, Clostridium perfringens, Burkholderia mallei, Burkholderia pseudomallei, cryptosporidia microorganisms, Tularemia (Francisella tularensis), Brucellosis (Brucella species), Chlamydia psittaci (psittacosis). Coxiella burneti (Q fever), Rickettsia prowazaki (Typhus fever), Vibrio vulnificus, Vibrio enteralyticus, Vibrio fischii, Vibrio cholera, Enterococcus faecalis, Staphylococcus epidermidis, Staphylococcus aureus, Enterobacter aerogenes, Corynebacterium diphtheriae, Pseudomonas aeruginosa, Acinetobacter calcoaceticus, Klebsiella pneumoniae, Serratia marcescens, Candida albicans, Microsporum audouini, Microsporum canis, Alicrosporum gypseum, Trichophyton mentagrophytes var. mentagrophytes, Trichophyton mentagrophytes var. interdigitale, Trichophyton rubrum, Trichophyton tonsurans, Trichophyton verrucosum, and Epidermophytum floccosum, Streptococcus (including Strep A, B, C, G) filoviruses such as Ebola and Marburg viruses, naviruses such as Lassa fever and Machupo viruses, alphaviruses such as Venezuelan equine encephalitis, eastern equine encephalitis, and western equine encephalitis, rotaviruses, calciviruses such as Norwalk virus, and hepatitis (A, B, and C) viruses.

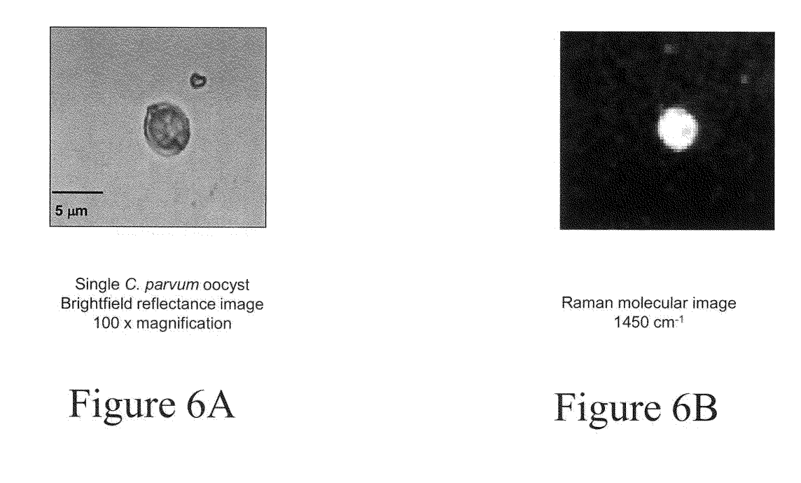

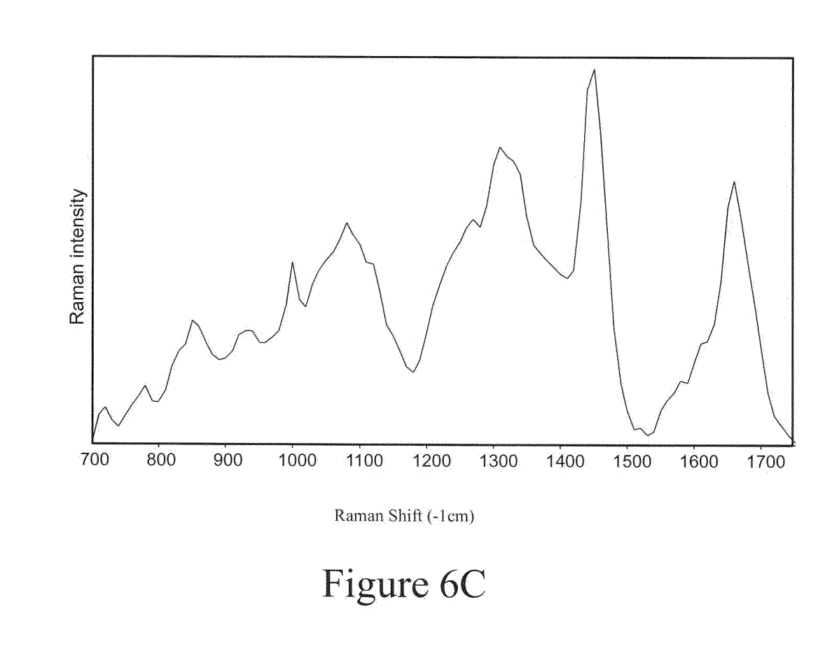

FIG. 6A illustrates a brightfield image of a single Single C. parvum oocyst. FIG. 6B illustrates a Raman chemical image of the Single C. parvum oocyst. FIG. 6C illustrates an image spectrum, obtained from a region of interest drawn around the cell, of the Single C. parvum oocyst. The system and method disclosed herein may be used to assess the presence of such particles in a sample. Such an analysis of similar particles may reveal of the presence of a bacteria and/or virus which may be indicative of a physiological characteristic.

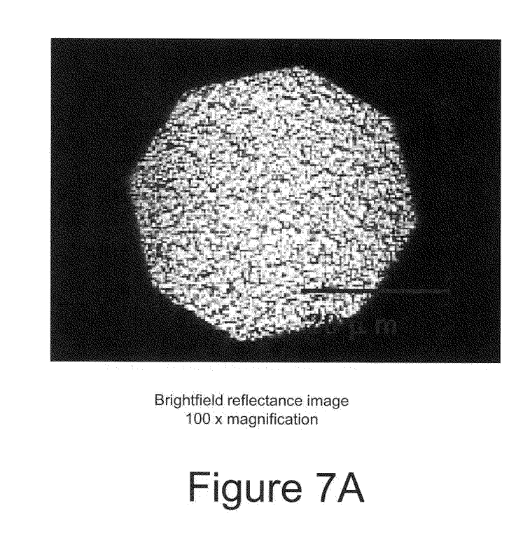

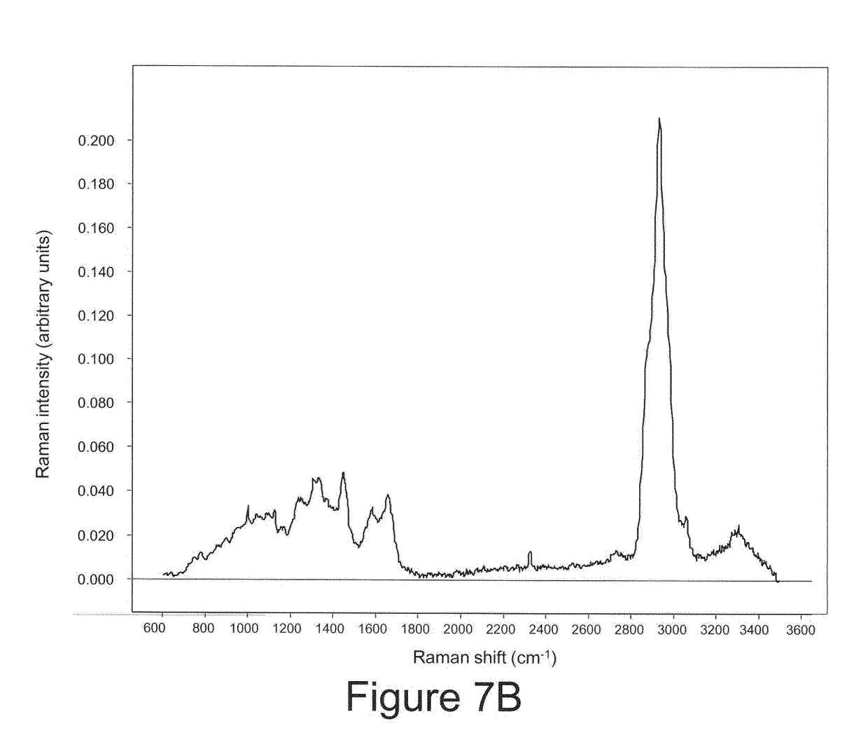

In one embodiment, a method of the present disclosure provides for the assessment of a sample comprising exhaled particles, further comprising the application of at least one chemometric technique. Such detection capabilities are illustrated by FIG. 7A-FIG. 9.

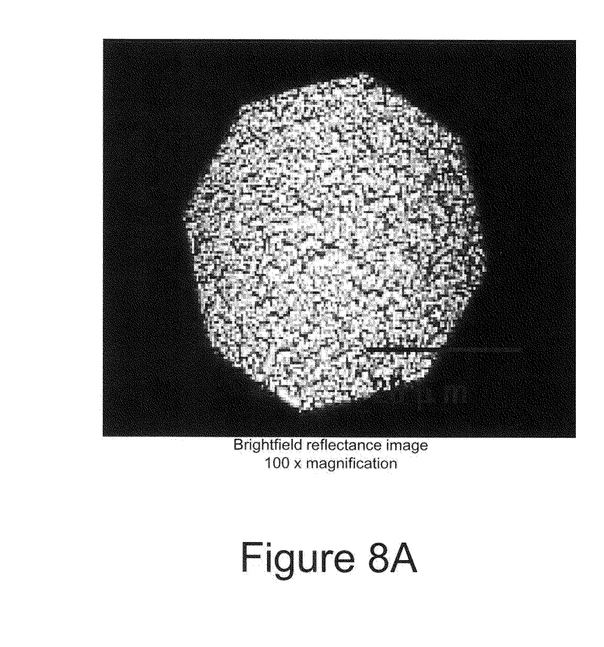

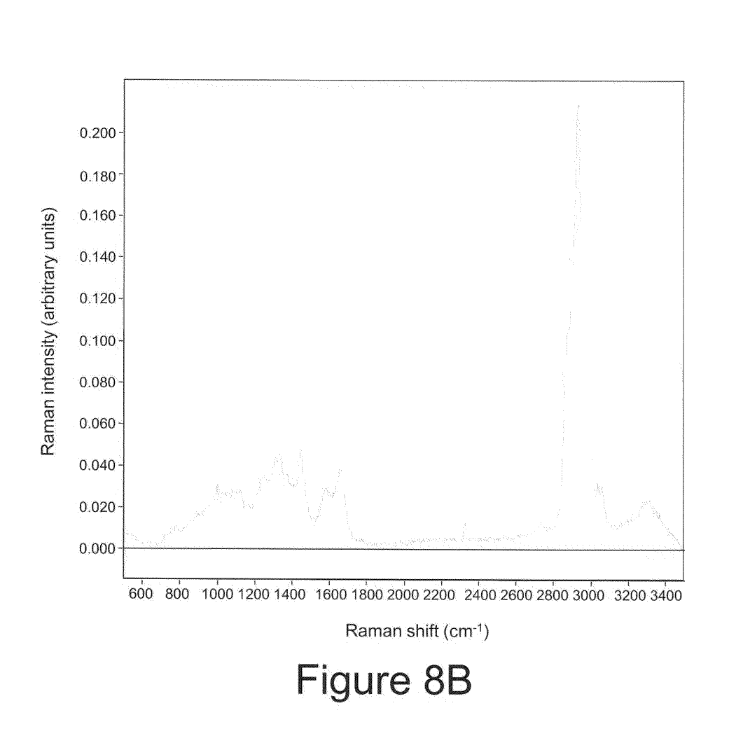

FIG. 7A is illustrative of a bright field reflectance image of a sample comprising Methicillin Resistant Staphylococcus Aureus (MRSA). FIG. 7B is representative of an average dispersive spectrum of MRSA. FIG. 8A is illustrative of a bright field reflectance image of a sample comprising Methicillin Sensitive Staphylococcus Aureus (MSSA). FIG. 8B is representative of an average dispersive spectrum of MSSA.

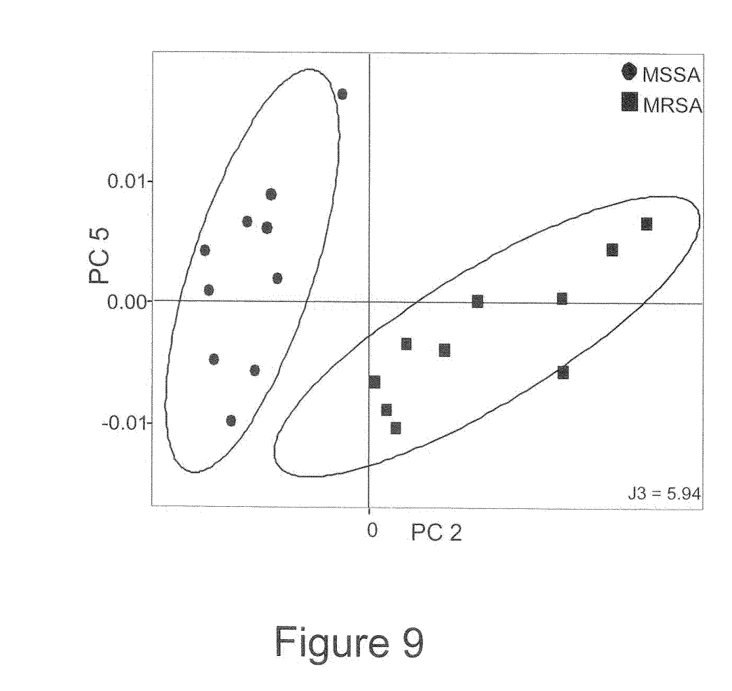

FIG. 9 represents principle component scores scatter plot of MSSA and MRSA dispersive spectra over the spectral region 700-1800 cm⁻¹. The data contained in the two different classes are clustered and completely separated from each other. This indicates that there are consistent differences in the spectra and therefore Raman spectroscopy is capable of distinguishing between two different subclasses of the same bacterial species. As illustrated by FIG. 9, a method of the present disclosure can be applied to interrogate a sample comprising exhaled particles to assess at least one physiological characteristic.

Although the disclosure is described using illustrative embodiments provided herein, it should be understood that the principles of the disclosure are not limited thereto and may include modification thereof and permutations thereof.

What is claimed is:

1. A method comprising:
depositing a sample comprising exhaled particles onto a substrate;
generating at least one autofluorescence image representative of the sample;
analyzing the autofluorescence image to target at least one region of interest which exhibits autofluorescence characteristic of at least one exhaled particle;
interrogating at least one region of interest to generate a test data set comprising at least one of: a hyperspectral image, a spectrum, and combinations thereof; and
analyzing the test data set to classify the sample as being associated with at least one physiological condition.

2. The method of claim 1 wherein the sample further comprises a bioaerosol sample.

3. The method of claim 1 further comprising introducing the sample into an aerosol collection device comprising the substrate.

4. The method of claim 3 further comprising transferring the sample from the aerosol collection device to a hyperspectral imaging device.

5. The method of claim 1 wherein the physiological condition comprises at least one of: a disease state, a metabolic state, an inflammatory state, an immunologic state, an infectious state, and combinations thereof.

6. The method of claim 1 wherein the test data set comprises at least one of: a fluorescence test data set, a Raman test data set, an infrared test data set, a visible test data set, an ultraviolet test data set, a LIBS test data set, and combinations thereof.

7. The method of claim 1 wherein generating at least one autofluorescence image comprises:
illuminating the sample to generate a plurality of interacted photons:
filtering the plurality of interacted photons; and
detecting the plurality of interacted photons to generate an autofluorescence image.

8. The method of claim 7 wherein filtering the plurality of interacted photons further comprises passing the plurality of interacted photons through a fiber array spectral translator device.

9. The method of claim 1 wherein interrogating at least one region of interest comprises:
illuminating a region of interest to generate a plurality of interacted photons;
filtering the plurality of interacted photons; and
detecting the plurality of interacted photons to generate the test data set.

10. The method of claim 9 wherein filtering the plurality of interacted photons comprises passing the plurality of interacted photons through a fiber array spectral translator device.

11. The method of claim 9 wherein filtering the plurality of interacted photons comprises passing the plurality of interacted photons through a filter comprising at least one of: a liquid crystal tunable filter, a multi-conjugate liquid crystal tunable filter, an acousto-optical tunable filter, a Lyot liquid crystal tunable filter, an Evans split-element liquid crystal tunable filter, a Sole liquid crystal tunable filter, a ferroelectric liquid, and combinations thereof.

12. The method of claim 1 wherein analyzing the test data set comprises comparing the test data set to at least one reference data set associated with at least one of: a known disease state, a known metabolic state, a known inflammatory state, a known immunologic state, a known infectious state, and combinations thereof.

13. The method of claim 12 wherein comparing the test data set to a reference data set comprises applying at least one chemometric technique.

14. The method of claim 13 wherein the chemometric technique comprises at least one of: principle component analysis, linear discriminant analysis, partial least squares discriminant analysis, maximum noise fraction, blind source separation, band target entropy minimization, cosine correlation analysis, classical least squares, cluster size insensitive fuzzy-c mean, directed agglomeration clustering, direct classical least squares, fuzzy-c mean, fast non negative least squares, independent component analysis, iterative target transformation factor analysis, k-means, key-set factor analysis, multivariate curve resolution alternating least squares, multilayer feed forward artificial neural network, multilayer perception-artificial neural network, positive matrix factorization, self modeling curve resolution, support vector machine, window evolving factor analysis, and orthogonal projection analysis.

15. The method of claim 1 wherein interrogating at least one region of interest comprises interrogating a plurality of regions of interest simultaneously.

16. The method of claim 1 wherein interrogating at least one region of interest comprises interrogating a plurality of regions of interest sequentially.

17. A system, comprising:
a processor; and
a non-transitory processor-readable storage medium in operable communication with the processor,
wherein the storage medium contains one or more programming instructions that, when executed, cause the processor to perform the following:
generate at least one autofluorescence image representative of the sample comprising exhaled particles,
analyze the autofluorescence image to target at least one region of interest which exhibits autofluorescence characteristic of at least one exhaled particle,
interrogate at least one region of interest, to generate a test data set comprising at least one a hyperspectral image, a spectrum, and combinations thereof, and
analyze the test data set to classify the sample as being associated with at least one physiological condition.

18. The system of claim 17, wherein the storage medium further contains one or more programming instructions that, when executed, cause the processor to compare the test data set to at least one reference data set associated with at least one of: a known disease state, a known metabolic state, a known inflammatory state, a known immunologic state, a known infectious state, and combinations thereof.

19. The system of claim 18 wherein the storage medium further contains one or more programming instructions that, when executed to compare the test data set to at least one reference data set, further cause the processor to apply at least one chemometric technique.
